(19) United States
(12) Patent Application Publication
(10) Pub. No.: US 2007/0053862 A1
(43) Pub. Date: Mar. 8, 2007

(54) Monoi Butter, Novel Cosmetic and/or Dermatological Compositions Comprising Monoi Butter and Uses Thereof

(76) Inventors: Yves Touboul, Aubagne (FR); Olivier Touboul, Aubagne (FR)

Correspondence Address: HEDMAN & COSTIGAN P.C., 1185 AVENUE OF THE AMERICAS, NEW YORK, NY, 10036, US

(21) Appl. No.: 10/545,589

(22) PCT Filed: Feb. 25, 2003
(86) PCT No.: PCT/FR03/00605

§ 371 Date: Aug. 23, 2006

(51) Int. Cl. A61K 8/97; A61K 36/18

(52) U.S. Cl. 424/74; 424/63; 424/64; 424/725

(57) ABSTRACT

Life's necessities, more particularly to cosmetics, hygiene and dermatology. The invention more specifically relates to a novel active cosmetic ingredient, a hydrogenated Monoï derivative, preferably from Tahiti, known as Monoï butter, having improved texture and a moisturizing, protecting, sanitizing, smoothing and softening effect. The invention also relates to the use of Monoï butter, more generally copra butter, in cosmetic and/or dermatological compositions, optionally in association with or mixed with one or several appropriate vehicles or excipients used in cosmetics or in dermatology to produce compositions having improved application properties.

The present invention relates to essentials of life, and more specifically to cosmetics and dermatology.

The present invention relates more particularly to a new cosmetically active compound, having moisturizing, protecting, satinizing, smoothing and softening properties, and the cosmetic compositions including this compound. These composition are intended for care and protection of skin—including lips and integument (nails, hair . . . )—as well as for sun care, make-up . . . .

The new compound of the present invention, called Monoï Butter, is a hydrogenated derivative of Monoï, preferably from Tahiti.

Monoï is a natural beauty product obtained by soft and natural maceration of copra oil with Tiare flowers.

Copra oil is extracted from the dried almond of palm tree (Cocos nucifera). After refining, it is macerated along with Tiare flowers (Gardenia tahitensis) according to a specific protocol.

Because of its moisturizing properties, Monoï, especially Tahiti Monoï, is widely used in various cosmetic compositions (creams, shampoos, after-sun lotions . . . ) for skin, nails and hair care.

The exceptional properties of Tahiti Monoï have been officially acknowledged in April 1992 by a Label of Origin, guarantee of its authenticity and quality. The decree no° 92-340 of the 1 Jan., 1992, clarifying the terms of use of the label, has been published in the Official Journal of French Republic (2 Apr. 1992, p. 4727 to 4729). The label “Tahiti Monoï” is specific to products from Tahiti, containing at least 90% copra oil macerated with Tiare flowers. On the other hand, the term “oil of Tahiti Monoï” refers to products containing around 50% Tahiti Monoï, usually admixed with another oil.

The key step of Monoï preparation is maceration, during which copra oil enriches itself slowly with various components, especially organic compounds. The Glucid and Natural Products Valuation Center published a study in July 2002, including the compared aromatic profiles of copra oil and Tahiti Monoï. These results, presented as Example 1, show that maceration confers particularly interesting properties to Monoï.

Application of cosmetic preparations including copra oil, and especially Monoï, leads to good moisturization, softening and better protection of epidermis and epidermal formations. Yet, the fluid and greasy texture of such preparations make them difficult and unpleasant to apply. Moreover, the rancid smell of ageing copra oil and Monoï requires adding large amounts of perfume or antioxidants in the cosmetic preparations. These compounds are likely to generate allergic reactions, especially when associated with sun exposure.

Technical answers have already been provided against copra oil ageing. According to document FR-A1-2698786, the adding of 10% wt. mineral, vegetal, animal or synthetic wax and vegetal butter stabilizes Tahiti Monoï. However, such a composition does not avoid a very greasy texture, either too solid or too fluid. According to document FR-A1-2405988, Monoï can be stabilized by adding a whole fresh Tiare flower in the container. Unfortunately, this solution cannot be used when Monoï is incorporated in a cream.

Thus, texture and stability problems of composition including copra oil, especially Monoï, had never been solved in a satisfactory way for cosmetics. This lack of solution restricted significantly the potential use of these compounds in cosmetics and dermatology.

The aim of this invention was then to improve the texture and stability of compositions including Monoï while retaining its particular characteristics such as moisturizing properties.

This double problem has been surprisingly solved by using a new compound, instead of Monoï, in cosmetic compositions. This compound, called Monoï Butter, is obtained through catalytic hydrogenation of Monoï. Unlike the oil, it cannot be oxidized. It has numerous other properties, as stability to heat, which allows it to be added to cosmetic preparations at high temperature, without alteration. It is also more resistant to storage. Keeping all the distinctive qualities of Tahiti Monoï, Monoï Butter is included in the protection given by the label of origin “Tahiti Monoï”.

A short existing publication refers to a compound called “Monoï Butter”, included in a cosmetic composition called Melvita, used to enhance the evenness of tan. Presenting a better texture than Monoï this compound is obtained by adding shea butter to Monoï.

On the contrary, Monoï Butter according to the invention contains only the hydrogenation product of Monoï. That is to say, it is constituted of the hydrogenated mixture of glycerids, free fatty acids and all the other compounds usually constituting Monoï.

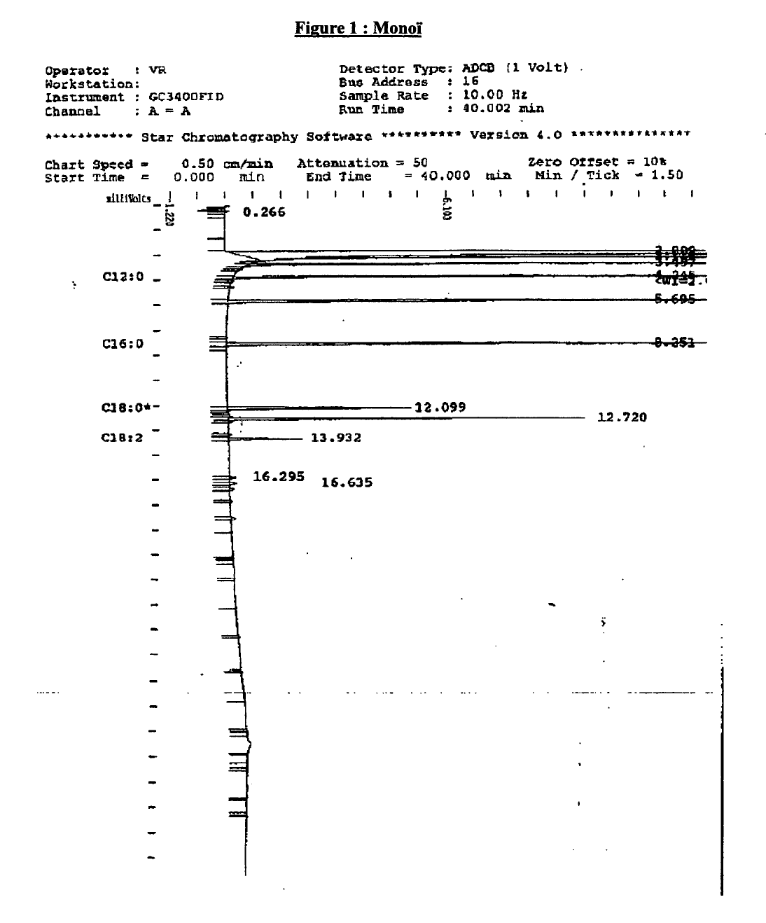

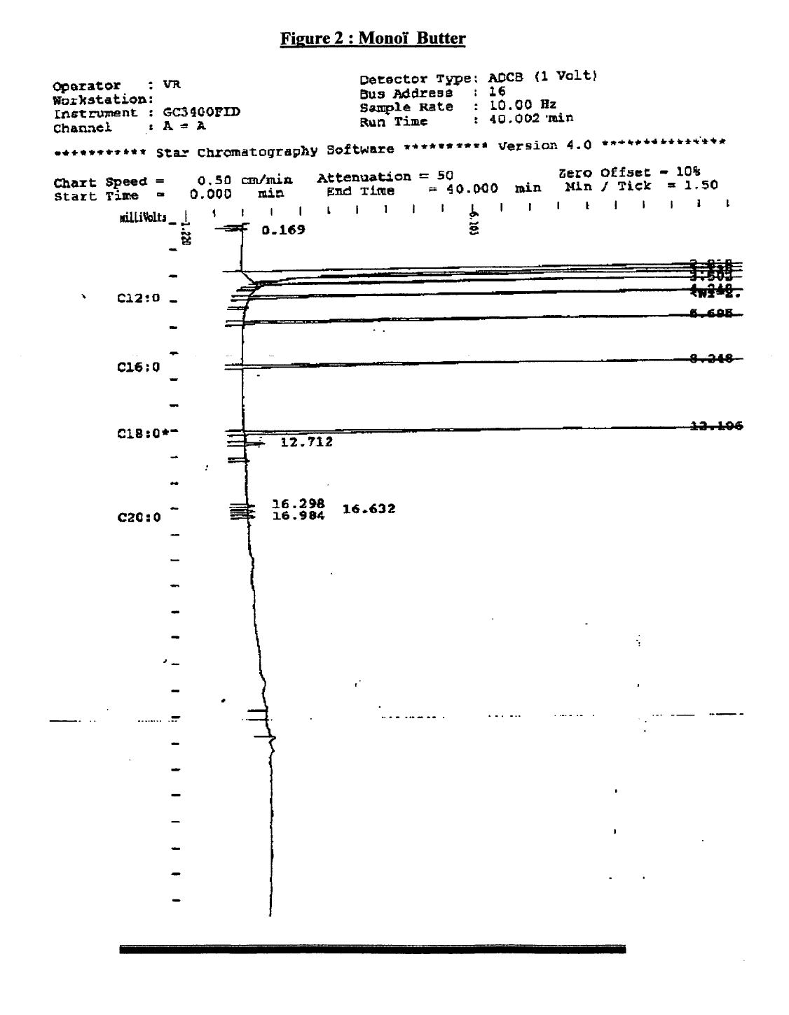

The free fatty acid composition of Monoï Butter has been determined by Gas Chromatography, the conditions being adapted to methyl ester analysis. The samples have been previously submitted to esterification in acid methanol. According to the usual method (NF ISO 5508 standard), weight percentages of the main fatty acid of Monoï Butter have been determined by comparison with an internal reference. The results are given in Table I. The chromatograms of Monoï and Monoï Butter are displayed in FIGS. 1 and 2.

TABLE I
Average fatty acid composition of Monoï Butter according
to the invention:

| Fatty acid | Average value (%) |
|---|---|
| Caprylic acid C8:0 | 8.1 |
| Capric acid C10:0 | 6.7 |
| Lauric acid C12:0 | 48.9 |
| Myristic acid C14:0 | 17.3 |
| Palmitic acid C16:0 | 8.4 |
| Stearic acid C18:0 | 10.4 |

At room temperature, Monoï Butter according to the invention is a tasteless, odourless ivory-white wax. Table II presents the physical state of Butter depending on temperature.

TABLE II
Physical state of Monoï and Monoï Butter depending on temperature:

| | Physical state* | Physical state* |
|---|---|---|
| Temperature (° C.) | of Monoï | of Monoï Butter |
| 20 | Amber solid | White solid |
| 34.5 | Clear liquid | Soft, slightly running paste |
| 35.5 | Clear liquid | Running and unclear |
| 37 | Clear liquid | Unclear liquid |
| 40 | Clear liquid | Clear liquid |

*Physical state determined by sensory observation.

Monoï Butter is characterized by a melting point of 34-38° C., determined by the adapted ISO 3104 method. Its relative density at 40° C., determined with densimeter ASTM 306H, is 0.906-0.911 kg/L. Insoluble in water and ethanol, it is soluble in oils at 40° C. Its iodine index is around 0.3 g/100 g, its acid index around 5 mg/g, its saponification index 258 mg_{KoH}/g, its peroxide index under 1 meqo₂/g. It is free of aerobic germ, mildew and yeast.

TABLE III
Physicochemical characteristics of Tahiti Monoï and Monoï Butter.

| Characteristics | Tahiti Monoï* | Tahiti Monoï Butter |
|---|---|---|
| Relative density (kg/L) | 0.910-0.920 | 0.906-0.911 |
| | (20° C.) | (40° C.) |
| Melting point (° C.) | 24-26 | 34-38 |
| Iodine index (gI₂/100 g) | 5-20 | <0.5 |
| Acid index (mg/g) | <5 | <5 |
| Saponification index (mg_{KOH}/g) | 240-270 | 240-270 |
| Peroxide index (meqO₂/kg) | <10 | <1 |

*as described in the “Tahiti Monoï” Label of Origin standards, JORF Apr. 2, 1992, p. 4727-4729.

Monoï Butter according to the invention combines the properties of Tahiti Monoï and of a butter. As Tahiti Monoïit brings a progressive and lasting moisturizing of the epidermis upper layers. It is active in hair mending, skin protection and micro-relief smoothing, and is well-tolerated. Its fluidity degree is more appropriate and its touch more pleasant than these of Tahiti Monoï. Cosmetic compositions containing it are easier to apply, non-sticky and do not get rancid. Moreover, Monoï butter is odourless, easily incorporable into cosmetic preparations and easier to emulsify, especially at high temperatures.

Procedure:

Monoï is elaborated along standard procedures:copra oil is extracted through high-temperature pressure, refined and macerated along with Tiare flowers. According to Decree n° 92-340 about the Tahiti Monoï Label of Origin, maceration has to last 10 days or more, with at least 10 flowers/L, having been picked as buds. After a usual 24-hour decantation, oil is filtered and antioxidant may be added. A more detailed protocol is to be found in the quoted decree.

Monoï Butter is then obtained by Monoï catalytic hydrogenation. This step leads to saturation of the unsaturated fatty acids contained by copra oil and Monof. A small amount of phytosterols also included in Monoï are unattacked by this reaction.

Hydrogenation may be carried on at high temperature and under oxygen-free atmosphere, along the following procedure: Monoï is dissolved in an inert solvent, for example an alkyl ether such as isopropyl ether. A hydrogenation catalyst on inert support is then added. This catalyst can be selected among platinum, platinum oxide, palladium and nickel, and the support among carbon, calcium carbonate and barium sulphate. The autoclave is purged with nitrogen until removal of air, and then heated under a pressurized hydrogen stream. The exit pressure of the gas is controlled. The reaction is considered complete when it is equal to the entry pressure, showing that no more hydrogen is consumed. The solvent is then removed through distillation or evaporation. The obtained wax-like mass is purified, filtered, and may be enriched with perfume and/or antioxydants such as butylhydroxyanisole (B.H.A.) or terbutylparacresol.

Monoï Butter according to the invention may be used, pure or perfumed, for direct application or incorporation in a cosmetic preparation. Examples of perfumes are Tiare, Coconut, Vanilla and Frangipani scents.

The invention also relates to cosmetic and/or dermatological compositions designed for skin moisturizing, including copra Butter, especially Monoï Butter preferably from Tahiti, admixed with one or more suitable excipient. Such compositions can include the Butter in an amount ranging from around 0.3 to 99% by weight, depending on the intended use. From around 2% and even 0.5% of Butter, the moisturizing and application condition are optimal. The amount of Monoï shall then depend on the desired consistency. For information, preferential ranges of Monoï amount for different uses are displayed in Example 2. In order to combine optimal fluidity and good moisturizing properties, body hygiene product will preferably include 50 to 80% Monoï Butter for bath oil, 0.3 to 1% for shampoos and 0.5 to 30% for soaps.

The compositions according to the invention are in a form suitable to skin and/or integument application, such as: gel; lotion, especially capillary lotion and varnish; emulsion or dispersion, especially oil/water and water/oil emulsion; cream, such as mascara, ointment, milk, foam; stick, such as lipstick and lip balm. When shaped as a cream, milk or gel, the composition includes preferably from 5 to 20% Monoï Butter by weight. When shaped as a balm or stick, especially for lips, it includes preferably from 10 to 60% Monoï Butter by weight.

According to the invention, the composition may also include sun-protective agents, especially for the manufacturing of sun care products. Well-known organic and/or mineral filters can be used as agents, such as metal silicate or metal oxide microsized particles. The metal can be selected among titanium, iron, cerium, aluminium . . . . The concentration and physicochemical characteristics of the selected filters depend on the desired final consistency of the composition.

According to the invention, the composition may also include suitable and appropriate inert cosmetic and/or pharmaceutical excipients, such as lipophilic or hydrophilic gelling agents, hydrophilic and lipophilic agents, gums, resins, tensioactives, solvents, charges like rice starch, preservatives, essential oils, antioxidants, pigments, colouring agents, mother-of-pearls, flakes, perfumes, odour absorbers, pH regulatory agents, fixatives and binders, selected among the compounds currently used in this kind of preparations.

The desired properties and consistency of the composition according to the invention will of course determine the choice and/or quantity of the complementary ingredients. The classical amount of an additive in a cosmetic preparation is between 0.01 to 20% by weight.

According to the invention, suitable hydrophilic gelling agents are carboxyvinyl polymers (carbomers), polysaccharides, polyacrylamides, acrylate/alkylacrylate copolymers, natural gums and clays.

According to the invention, suitable lipophilic gelling agents are polyethylenes, fatty acid metal salts and modified clays such as bentones.

According to the invention, examples of hydrophilic and lipophilic agents are polyols such as glycerol, propyleneglycol, vitamins, moisturizing agents such as Laurydone® (Bioeurope) or allantoin, emollients, peeling agents such as coco and apricot powder, antioxidants or anti-radical agents such as β-carotene, anti-inflammatory drugs such as anthranilic acid derivatives, soothing agents and mixtures of these compounds.

Lipidic vesicles commonly used in cosmetics may also be included in the composition.

A composition containing hydrogenated Monoï, especially from Tahiti, may also include other hydrogenated oils.

When the composition according to the invention is an emulsion, it includes commonly used emulsifiers and co-emulsifiers. Examples of suitable emulsifiers and co-emulsifiers are fatty acid glycerol esters such as glyceryl stearate, or fatty acid polyethylene glycol esters such as PEG-20 stearate. The emulsifier and co-emulsifier is preferably included in an amount between 0.3 and 30% by weight, most preferably from 0.5 to 5%.

According to the invention, examples of suitable colouring agents are β-carotene, Soudan brown, DC Red 17, quinoline yellow.

According to the invention, examples of suitable mother-of-pearls are micas, covered with oxides or with natural pigments, or cholesterol esters.

According to the invention, examples of suitable pigments are mineral pigments as zinc, iron and cerium oxides, and organic pigments as carbon black and commonly used lakes.

According to the invention, the composition can be made along usual standards of cosmetology and dermatology. Unlike copra oil, which can be incorporated at 26° C., Monoï Butter should be first warmed up to 40° C. in order to make incorporation into the composition easier.

The invention also relates to the use in cosmetics of compositions including copra Butter, especially Monoï Butter preferably from Tahiti, for man and animal epidermis and/or integument (nails, hairs . . . ) care and/or treatment and/or protection.

The composition has to be preferably used for skin, lips, mucosa and/or integument (eyebrows, eyelashes . . . ) make-up, especially when shaped as lipstick, eye shadow, mascara . . . .

Compositions according to the invention has to be used in cosmetics for tegument moisturizing, smoothing, softening and mending. It shall also be used in dermatology, for the hydrolipidic film restoration of dehydrated tegument and for protection against environmental stress such as climate (cold, wind, sunburns), burns and some therapeutic treatments.

Most preferably, such compositions have to be used in dermatology, to prevent and/or cure skin and/or integument cracking and drying.

Thus, according to the invention, copra Butter, especially Monoï Butter preferably from Tahiti may be applied for the production of a dermatological composition usable on skin and/or integument, to prevent and/or cure skin—especially skin folds—and/or integument drying.

Such compositions may be used on man or animal.

The oily consistency of Monoï having until now prevented its use in this area, Monoï Butter would be particularly fitted for use in domestic supplies, where its Tiare flower smell would be appreciated. Examples of use are household cleaning supplies, perfumed candles, incense . . . .

Compositions according to the invention are neither toxic nor allergenic nor displaying local intolerance.

The following examples are given for information, and shall not be considered as limits to the invention. Concentrations are expressed in percentage of the composition global weight.

### EXAMPLE 1

### Compared Aromatic Profiles of Copra Oil and Tahiti Monoï

Compounds have been extracted by Solid Phase Micro-Extraction (SPME), using a 100 μm polydimethylsiloxane (PDMS) fiber, specific to volatile compounds. Desorption time is 4 min. SPME extraction was made at 35° C. during 45 min. Compositions were then determined through GC/MS.

The aromatic profile study of Tahiti Monoï 220 flowers/L only means to make easier identification of the “traditional” Tahiti Monoï compounds.

| Retention | | Copra oil | Tahiti Monoï | Tahiti Monoï |
|---|---|---|---|---|
| time (min) | Compound | (%)(¹) | 10 flowers/L (%)(¹)(²) | 220 flowers/L (%)(¹)(²) |
| 9.509 | Cis-3-hexenol | | 0.63 | 6.13 |
| 10.311 | Hexanol | | 0.81 | 0.57 |
| 11.387 | 2-heptanone | | 57.87 | 6.91 |
| 13.508 | Methyl hexanoate | | 0.47 | 0.79 |
| 17.216 | Hexanoic acid | 9.23 | | |
| 18.530 | Ethyl hexanoate | | 1.79 | 1.61 |
| 18.897 | Ethyl hex-3-enoate | | | 0.42 |
| 20.942 | Benzylic alcohol | | | 1.17 |
| 21.612 | Phenylacetaldehyde | | | 1.75 |
| 24.619 | Linalool oxide | | | 0.57 |
| 25.038 | 2-nonanone | 1.66 | | |
| 25.174 | Methyl benzoate | | | 1.87 |
| 25.637 | Linalool | | 0.87 | 13.72 |
| 26.448 | Phenyl ethyl alcohol | | 0.82 | 2.61 |
| 27.407 | Methyl caprylate | | 4.62 | 5.51 |
| 30.116 | Benzyl acetate | | | 0.25 |
| 30.606 | Ethyl benzoate | | 0.70 | 13.16 |
| 31.435 | Octanoic acid | 54.63 | | |
| 32.080 | Methyl salicylate | | 0.68 | 15.19 |
| 32.586 | Ethyl caprylate | | 12.38 | 6.52 |
| 35.673 | Phenyl ethyl acetate | | | 0.65 |
| 36.552 | γ-octalactone | | 1.75 | |
| 37.342 | Ethyl salicylate | | | 0.48 |
| 38.279 | δ-octalactone | | 0.37 | |
| 38.949 | Indole | | | 0.55 |
| 39.124 | 2-undecanone | 34.47 | | |
| 41.191 | Methyl decanoate | | 1.90 | 0.96 |
| 42.553 | Benzyl butyrate | | | 0.24 |
| 45.821 | Ethyl decanoate | | 5.79 | 3.02 |
| 52.444 | Cis-α-bergamotene | | | 0.21 |
| 53.691 | Methyl dodecanoate | | 2.54 | 3.49 |
| 56.340 | Cis-3-hexenyl | | 0.27 | 8.17 |
| | benzoate | | | |
| 57.776 | Ethyl laurate | | 5.76 | 3.47 |

(¹)BHA included in the three samples was not considered. A detection rate of 1 is admitted in each case.
(²)Compounds included in copra oil are found in same proportions in Monoï. Thus, relative percentages of Monoï compound have been evaluated apart from the compounds found in copra oil.

Identified fatty acids correspond to acids naturally included in copra oil.

Volatile compounds found in labelled Tahiti Monoï 10 flowers/L bring fruit notes as banana (2-heptanone), pineapple (methyl and ethyl hexanoate), pear/apple (methyl and ethyl caprylate), green notes (hexanol, cis-3-hexanol), floral notes (ethyl benzoate) wine notes, rose notes (phenyl ethyl alcohol), bergamot, coriander (linalool), waxy and greasy notes (ethyl laurate).

### EXAMPLE 2

### Average Advised Concentrations Depending on the Product Type

This table indicates minima and maxima advised concentrations of Tahiti Monoï Butter according to the invention. The minima conditions have been determined according to the Label of Origin standards.

| | | Minima | Maxima |
|---|---|---|---|
| | Advised concentrations | concentrations | concentrations |
| Product type | (%) | (%) | (%) |
| Suntan | Oils: 50 to 99 | 50 | 100 |
| | Gels: 0.5 to 2 | 1 | 5 |
| | Balms: 20 to 95 | 1 | 95 |
| | Emulsions: 5 to 20 | 1 | 20 |
| Make-up | Oils: 10 to 50 | 2 | 50 |
| | Balms: 10 to 40 | 2 | 40 |
| | Emulsions: 5 to 20 | 2 | 25 |
| Hygiene | Gels: 0.5 to 1 | 0.3 | 1 |
| | Bath oils: 50 to 80 | 50 | 100 |
| | Shampoos: 0.3 to 1 | 0.3 | 1 |
| | Soaps: 5 to 30 | 30 | 80 |
| Hair | Oils: 10 to 70 | 1 | 100 |
| | Balms: 10 to 95 | 1 | 100 |
| | Emulsions: 5 to 20 | 1 | 20 |
| Skin | Balms: 10 to 95 | 10 | 95 |
| | Emulsions: 5 to 20 | 1 | 20 |
| | Gels: 0.5 to 1 | 1 | 5 |
| Lips | Sticks: 10 to 30 | 2 | 50 |
| | Balms: 30 to 60 | 2 | 70 |

### EXAMPLE 3

### Dermatological/Cosmetic Formulations

Unless otherwise specified, products under registered trademark are marketed by the firm SEPPIC.

a) Moisturizing lotion with Monoï Butter:

| | Composition (%) |
|---|---|
| Phase A | |
| Montane ® 60 | 2.00 |
| Montanox ® 60DF | 2.00 |
| Monoï Butter | 5.00 |
| Lanol ® 99 (Pharmastra S.A.) | 5.00 |
| Phase B | |
| Demineralized water | s.q. 100 |
| Sepigel ® 305 | 2.00 |
| Phase C | |
| Sepicide ® Cl | 0.20 |
| Sepicide ® HB | 0.30 |
| Perfume | 0.80 |

Procedure:
1) Phase A constituents are mixed and heated to 65° C.
2) Phase B is heated to 45° C., phase A is added and the mixture is emulsified.

3) Phase C is added to the emulsion at 35° C.

b) Monoï Butter Cream (soft):

| | Composition (%) |
|---|---|
| Phase A | |
| Montanove ® 202 | 5.00 |
| Monoï Butter | 10.00 |
| Lanol ® 99 (Pharmastra S.A.) | 5.00 |
| Phase B | |
| Demineralized water | s.q. 100 |
| Sepigel ® 305 | 1.50 |
| Phase C | |
| Kukui oil | 2.00 |
| Sepicide ® Cl | 0.20 |
| Sepicide ® HB | 0.30 |
| Phase D | |
| Perfume | 0.80 |
| DL-α-Tocopherol | 0.05 |

Procedure:
1) Phase A constituents are mixed and heated to 80° C.
2) Phase B is heated to 45° C., phase A is added and the mixture is emulsified.
3) Phase C is added to the emulsion at 35° C.

4) Phase D is then added.

c) Polynesian oil and Monoï Butter Cream:

| | Composition (%) |
|---|---|
| Phase A | |
| Montanox ® 68 | 5.00 |
| Monoï Butter | 10.00 |
| Phase B | |
| Demineralized water | s.q. 100 |
| Sepigel ® 305 | 1.00 |
| Phase C | |
| Tamanu oil | 2.00 |
| Candlenut oil | 3.00 |
| Sepicide ® Cl | 0.20 |
| Sepicide ® HB | 0.30 |
| Phase D | |
| Perfume | 0.80 |
| DL-α-Tocopherol | 0.05 |

Procedure:
1) Phase A constituents are mixed and heated to 70° C.
2) Phase B is heated to 45° C., phase A is added and the mixture is emulsified.
3) Phase C is added to the emulsion at 40° C.

4) Phase D is then added.

d) Monoï Butter Cream Gel (soft):

| | Composition (%) |
|---|---|
| Phase A | |
| Simulgel ® EG* | 3.00 |
| Monoï Butter | 5.00 |
| Lanol ® 1681 (Pharmastra S.A.) | 3.00 |
| Phase B | |
| Demineralized water | s.q. 100 |
| Phase C | |
| Sepicide ® Cl | 0.20 |
| Sepicide ® HB | 0.30 |
| Perfume | 0.80 |

*sodium acrylate, sodium dimethyl acryloyl taurate, isohexadecane polysorbate 80.

Procedure:
1) Phase A constituents are mixed and heated to a minimum temperature of about 40°
2) Phase B is heated to 40° C., phase A is added and the mixture is emulsified.

3) When the emulsion begins to cool, phase C is added.

e) Monoï Butter Cream (soft):

| | Composition (%) |
|---|---|
| Phase A | |
| Montanove ® 202 | 5.00 |
| Monoï Butter | 12.00 |
| Lanol ® 99 (Pharmastra S.A.) | 5.00 |
| Phase B | |
| Demineralized water | s.q. 100 |
| Sepigel ® 305 | 1.50 |
| Phase C | |
| Candlenut oil | 3.00 |
| Phase D | |
| Sepicide ® Cl | 0.20 |
| Sepicide ® HB | 0.30 |
| Perfume | 0.80 |
| DL-α-Tocopherol | 0.05 |

Procedure:
1) Phase A constituents are mixed and heated to 80° C.
2) Phase B is heated to 45° C., phase A is added and the mixture is emulsified.
3) Phase C is added to the emulsion at 40° C.

4) Phase D is then added.

f) Peeling Monoï Butter Cream:

| | Composition (%) |
|---|---|
| Phase A | |
| Lanol Wax ® CTO (Pharmastra S.A.) | 5.00 |
| Monoï Butter | 10.00 |
| Lanol ® 99 (Pharmastra S.A.) | 5.00 |
| Petrolatum oil | 5.00 |
| Phase B | |
| Demineralized water | s.q. 100 |
| Sepigel ® 305 | 1.00 |
| Phase C | |
| Coco powder | 5.00 |
| Allantoin | 0.20 |
| Phase D | |
| Sepicide ® Cl | 0.20 |
| Sepicide ® HB | 0.30 |
| Perfume | 0.80 |

Procedure:
1) Phase A constituents are mixed and heated to 70° C.
2) Phase B is heated to 45° C., phase A is added and the mixture is emulsified.

3) Phase C, then phase D are added to the emulsion.

g) Monoï Butter Balm:

| | Composition (%) |
|---|---|
| Phase A | |
| Carnauba wax | 5.00 |
| Bee wax | 5.00 |
| Monoï Butter | 59.40 |
| Phase B | |
| Thick petrolatum | 10.00 |
| Tahiti Monoï | 10.00 |
| Cosobiol ® (Laserson) | 10.00 |
| Phase C | |
| Natural Tocopherol | 0.20 |
| Perfume | 0.40 |

Procedure:
1) Phase A constituents are mixed and heated to 75° C.
2) Phase B constituents are mixed and heated to 50° C.
3) Phase B is added to phase A and the temperature is controlled.

4) When at 40-45° C., the mixture is added to phase C.

h) Monoï Butter lipstick:

| | Composition (%) |
|---|---|
| Phase A | |
| Carnauba wax | 6.00 |
| Synthetic wax | 5.80 |
| Bee wax | 9.00 |
| Monoï Butter | 11.00 |
| Phase B | |
| Thick petrolatum | 31.00 |
| Calendula phytosol | 9.00 |
| Cosobiol ® (Laserson) | 27.00 |
| Natural Tocopherol | 0.20 |
| Kathon ® (Rohm and Haas) | 0.10 |
| Allantoin | 0.50 |
| Perfume | 0.40 |

Procedure:
1) Phase A constituents are mixed and heated to a minimum temperature of 75° C.
2) Phase B constituents are mixed and heated to a minimum temperature of 50° C.
3) Phase B is added to phase A.
4) The hot mixture is poured in lipstick moulds and left to rest until solidification.

1. Monoï Butter, obtained by hydrogenation.

2. Monoï Butter of claim 1 wherein it is obtained from Tahiti Monoï, containing at least 90% copra oil having macerated along with Tiare flowers.

3. Monoï Butter of claim 1 wherein it is constituted of the hydrogenated mixture of glycerids, free fatty acids and all other usual constituents of Monoï, and its melting point is between 34 and 38° C.

4. A process to obtain Monoï Butter comprising dissolving Monoï in an inert solvent, adding a hydrogenation catalyst on inert support, hydrogenating at high temperature and under an oxygen-free atmosphere to obtain hydrogenation product Monoï Butter, having a melting point of 34-38° C.

5. (canceled)

6. A cosmetic and/or dermatological composition comprising copra butter obtained by hydrogenation of copra oil, associated or admixed with at least one suitable excipients and vehicles.

7. A cosmetic and/or dermatological composition to of claim 6, wherein the said butter in Monoï butter of claim 1, associated or mixed with one suitable excipients and vehicles.

8. A cosmetic and/or dermatological composition of claim 6 wherein it includes from 0.3 to 99% by weight of the said butter.

9. A cosmetic and/or dermatological composition of claim 6 wherein it is in a form selected from the group consisting of a gel, milk, lotion or cream and includes 5 to 20% by weight of the said butter.

10. A cosmetic and/or dermatological composition according to a of claim 6 wherein it is a balm or stick and includes 10 to 60% by weight of the said butter.

11. A cosmetic and/or dermatological composition of claim 6 wherein it also includes at least one hydrophilic or lipophilic gelling agent.

12. A cosmetic and/or dermatological composition of claim 6 wherein it also includes a sun-protective agent.

13. A cosmetic and/or dermatological composition of claim 6 wherein it also includes at least one additive selected selected from the group consisting of pigments and mother-of pearls.

14. (canceled)

15. A cosmetic and/or dermatological composition of claim 6 wherein it is designed as an oil/water or water/oil emulsion and includes emulsifiers and co-emulsifiers.

16-19. (canceled)

20. A method of caring or protecting skin comprising applying directly to the skin or epidermis a Monoï butter of claim 1.

21. The method of claim 20 the butter is in the form of claim 7.

22. The method of claim 20 wherein the skin is protected from integument drying.
